(19) United States
(12) Patent Application Publication
(10) Pub. No.: US 2004/0091932 A1
(43) Pub. Date: May 13, 2004

(54) Method for producing from microbial sources enzymes having multiple improved characteristics

(76) Inventors: Nugzar N. Nutsibidze, Urbandale, IA (US); Michelle P. Moore, Des Moines, IA (US); Friedhelm Brinkhaus, West Des Moines, IA (US); Peter Salamone, West Des Moines, IA (US); Michelle Fonda, Meadow Vista, CA (US); Matthew Paul Brown, Rocklin, CA (US); Karen Loser, Auburn, CA (US); Ingrid Gennity, Fresno, CA (US); Nicholas Everett, Meadow Vista, CA (US)

Correspondence Address: Kent A. Herink, The Financial Center, Suite 2500, 666 Walnut Street, Des Moines, IA, 50309-3993, US

(21) Appl. No.: 10/293,103

(22) Filed: Nov. 13, 2002

(51) Int. Cl. G01N 33/53; C12Q 1/34

(52) U.S. Cl. 435/7.1; 435/18

(57) ABSTRACT

A method of producing from sources of microorganisms enzymes having multiple improved characteristics. Soil samples are collected from a wide variety of sources that are diverse geographically and environmentally. The microorganisms are screened using microbiological screens to select microorganisms that produce enzymes that are active over a broad range of temperatures. Enzymes from selected microorganisms are screened for improved thermostability, extended active temperature range, extended active pH range, extended shelf-life, and improved protease resistance.

### BACKGROUND OF THE INVENTION

The invention relates generally to the production of enzymes with two or more improved characteristics and, more specifically, to a method for producing from microbial sources enzymes which have multiple improved characteristics, such as improved thermostability, extended active temperature range, extended active pH range, extended shelf-life, and improved protease resistance.

Enzymes are in wide use in a variety of industries and applications, including animal feeds, food processing, detergents, baking, and brewing. It is a well-accepted practice in the animal production industry to enhance the energy value of least-cost diets further by the use of hydrolytic enzymes as feed ingredients. The availability of dietary energy depends on the structure and quantity of carbohydrates in the cell wall of cereals. For example, the addition of carbohydrases (cellulase, hemicellulases and β-glucanase) to the poultry feed substantially enhances the availability of metabolizable energy and digestibility of cereals by poultry. The enzymes assist in the digestion and absorption of nutrients from lower quality feedstuffs by providing exogenous enzymes to the animal or supplementing its endogenous enzymes, and assist in the neutralization of antinutritional factors in feedstuffs, such as phytic acid, α-galactosides, and β-glucans. The efficient and economical use of enzymes in animal feed is still hampered by the fact that enzymes are heat labile and typically do not operate optimally under conditions encountered in the animal gastrointestinal tract. The application and use of these enzymes has been limited, also, due to their limited stability to extremes in pH, extended storage, presence of proteases, and so forth. It therefore is desirable to identify and develop enzymes with a combination of these properties combined in one gene in order to maximize the effect.

One approach to identifying enzymes with improved characteristics has been to collect microorganisms from extreme environments and test them for enzymatic activity. These so-called “extremophiles” have been found to exhibit activities under unusual conditions, e.g., high temperature or high pH, with stabilities under these extreme conditions that are much greater than previously found in biological systems. Researchers have discovered that certain extremophiles have novel metabolic pathways. Hough, D. W., Danson, M. J. Extremozymes. Curr. Opin. Chem. Biol. 1999, 3:39-46. The initial promise of these novel enzymes, however, has been tempered by the discovery that having stability and activity at an extreme condition does not necessarily mean that the enzyme will have stability and activity at the conditions under which the enzyme is used. For example, an enzyme isolated from an extremophile found in a high temperature environment, such as a geothermal pool, may produce an enzyme that remains stable and may display activity at temperatures approaching the boiling point of water. This enzyme, accordingly, would likely survive more extreme processing conditions that are commonly used in the animal feed industry, such as extrusion, than would enzymes from conventional biological sources. The enzyme may not, however, have a sufficiently broad active temperature range to have a high level of activity at the body temperature of the animal to which it is to be fed.

Another approach to the development of improved enzymes has been through the use of directed evolution. Directed enzyme evolution typically begins with the production of a library of mutated genes using techniques such as DNA shuffling, error-prone PCR, and targeted cassette mutagenesis. Screening techniques are used to identify gene products which show improvement with regard to a desired property or number of properties. The genes encoding the selected gene products are subjected to further cycles of mutation and screening in an effort to accumulate desirable mutations. Because the number of possible variants under mutation increases rapidly with the size of the enzyme and the number of amino acids that are allowed to vary simultaneously, even a small protein can generate a number of variants that would be impossible to screen economically. In addition, since most mutations result in poorer, rather than improved, enzymes, a large number of mutations must be made to have a likelihood of identifying a sufficiently improved enzyme. Kuchner, O., Arnold, F. H. Directed evolution of enzyme catalysts. Trends in Biotech. 1997. 15: 523-530.

Genetic engineering and protein engineering techniques are also being applied in an attempt to produce improved enzymes. Unfortunately, the extensive knowledge of the relationships between protein sequences, structure and function that is required for rational protein engineering is available for only a very small fraction of known enzymes (Kuchner et al.).

There is a need, accordingly, for a method of identifying and producing enzymes that have improved characteristics that is both efficient and results in enzymes with not only improved performance optima, but also an improved or extended range of stability and activity. Such enzymes with multiple enhanced performance criteria will not only be able to survive harsh industrial conditions of temperature, pH, and the presence of solvents, but also will remain active and stable under storage conditions and in the digestive system of the animal. The present invention exploits the diversity of natural genes when used as starting materials as an alternative to the usually deleterious mutagenesis used in random and rational protein engineering strategies.

### SUMMARY OF THE INVENTION

Microorganisms having a large degree of genetic diversity are used as starting materials in the method of the present invention. The microorganisms originate in soil samples that have been collected from geographically and ecologically diverse environments. The samples are maintained in a storage facility to provide a sustainable library of such microorganisms. Isolates from the soil samples are selected for a specific activity using an appropriate screen. High performing isolates are selected for use in biochemical screening assays which test for activity and stability over a range of temperatures and pH, and for thermostability over extended periods of time. Enzymes having multiple features combined in one enzyme, such as temperature activity maxima higher than commonly found, pH activity minima lower than commonly found, improved thermostability, extended shelf-life, and high activity at typical animal body temperatures, this fitting the needs of specific applications, were identified.

The identification process involves choosing soil samples from non-extreme environments (but a wide range of geographic populations and ecological niches), isolating mesophilic microorganisms using a wide range of isolation media and selecting those organisms that looked unusual (to the experienced eye of microbiologists), were of low abundance, slow to appear on isolation plates, and/or grew well on media enriched with potential substrates of the enzyme(s) of interest. This combination favors selecting microorganisms that represent broad genetic diversity, potential novelty, and activity against the substrates of interest. After subculturing to produce pure cultures, selected microorganisms were grown in liquid broth culture in shake-flasks and the resulting crude culture broths tested for enzyme activity using appropriate substrates. Those cultures broths showing significant enzyme activity were then tested for heat stability. Those cultures showing significant enzyme activity after heat treatment were then evaluated further by performing temperature-activity profiles and pH-activity profiles and those that combined good temperature and pH performance were advanced into stability testing and purification and characterization of individual enzymes.

### BRIEF DESCRIPTION OF THE DRAWINGS

FIG. 1 is a a pie chart showing the geographic diversity of the soil samples used as the source of microorganisms used in the present application.

FIG. 2 is a chart of galactosidase activity over a range of temperatures for culture broths selected from culture broths screened for activity above 65° C.

FIG. 3 is a chart of galactosidase activity over a range of pH for culture broths selected from culture broths screened for activity above 65° C.

FIGS. 4a and 4b are temperature profile charts of galactosidase activity of two selected culture broths before and after heat treatment at 65° C. for 20 minutes.

FIG. 5 is a chart of galactosidase activity of selected culture broths over time at 65° C.

FIG. 6 is a pH profile chart of galactosidase activity of selected culture broths.

FIG. 7 is a chart of galactosidase activity of selected culture broths at pH 3.0 over time.

### DETAILED DESCRIPTION OF A PREFERRED EMBODIMENT

A successful approach to the discovery of natural products such as novel enzymes involves considering the desired product characteristics and then using ecophysiological methods of isolation and screening (Hunter, J. C., Fonda, M., Sotos, L., Toso, B., and Belt, A. 1984. Ecological approaches in isolation. Dev. Ind. Microbiol. 25:247-66). For a general discussion of the principles of the isolation of microbial cultures and detailed protocols, see Hunter, J. C. and Belt, A. 1999 “Isolation of Cultures” Chpt. 1 in: Manual of Industrial Microbiology and Biotechnology, eds. A. L. Demain and J. E. Davies, Second edition, ASM Press, pp. 3-20. Non-selective isolation conditions can be used to isolate diverse microorganisms from soil samples, and may be particularly useful for the isolation of cultures from ecological microenvironments that have already provided a selective advantage for the desired physiology (e.g. environments that contain the appropriate substrates for the desired enzyme activity, desired pH, exposure to high or low temperatures etc.). Alternatively, there are many ways to pretreat soils and use selective media to favor the growth of specific genera or cultures that are likely to possess a specific desired enzyme activity (ibid). This may include the incorporation of a substrate for the desired enzyme activity in either the isolation or screening media.

Fungi can be isolated from soil using a variety of methods (Hunter-Cevera and Belt, 1999). Some of these include dilution plating, pour plating, stamping, and implanting. Direct isolation from fruiting bodies and plant material can be used, but is time consuming and tedious. Enriching for a particular type of fungus through the addition of certain compounds or adding a substrate that elicits the desired enzyme production can also be employed. Once growth appears on the selective agar plates (1 to 6 weeks), the hyphae are aseptically transferred to a common maintenance medium. The purpose of this is two-fold: (1) to check for contamination (i.e. cultures that are not yet genetically pure) and (2) to dereplicate equivalent strains from the same source material. Axenic cultures can then be used for screening purposes. Plugs or blocks of hyphal growth are inoculated into appropriate screening broth(s) and shaken for 5 to 14 days or until production of desired compound/enzyme is achieved. Preservation of the strains via slanting and freezing should be performed at this time in order to retain the integrity and physiological capabilities of the original strain (Dahmen, H., Staub, T., and Schwinn, F. J., 1983. Technique for long-term preservation of phytopathogenic fungi in liquid nitrogen. Phytopathology 73: 241-246, Monaghan, R. L., Gagliardi, M. M., and Streicher, S. L. 1999. “Culture Preservation and Inoculum Development” in: Manual of Industrial Microbiology and Biotechnology, eds. A. L. Demain and J. E. Davies, Second edition, ASM Press, pp. 29-48) which can otherwise be lost during repeated subcultures. At the end of the fermentation period, a sample of the supernatant broth or culture extract is screened for the desired activity.

When the desired enzyme must contain multiple desired characteristics, these may be tested either simultaneously or, more often, sequentially. For example, cultures secreting a desired enzyme activity may be detected by incorporating an insoluble substrate into solid culture media, followed by observation of clearing zones (Shieh, T. R. and Ware, J. H. 1968. Survey of Microorganisms for the production of Extracellular Phytase. Appl. Microbiol. 16: 1348-1351). Alternatively, a substrate that generates a colored product may be used either in situ on the culture plates or to assay samples of culture broths or extracts. Wherever possible the substrate(s) used for screening should be as close as possible to the final intended substrate. An example of sequential screening would be to identify cultures producing a desired activity by a plate growth or clearing zone assay, characterize the temperature activity profiles and heat stabilities using crude extracts, characterize the pH activity profile of those candidates that pass the temperature stability tests, compare the activities of the remaining candidates under real-world conditions, and determine the novelty of the enzyme(s) sequences using either proteomics techniques or DNA sequence based techniques (DNA blots or gene sequencing).

### EXAMPLE 1

### Microbial Screening of Soil Samples

Fungal isolates from a library of soil samples obtained from geographically and ecologically diverse origins were screened for galactosidase activities that could survive a 65° C. heat treatment, that would have a broad temperature activity profile, and would have significant activity below pH 3.0. The fungal isolates were grown in a liquid medium that contained, per liter, 25 g soy flour, 1.0 g yeast extract, 0.7 g K₂HPO₄, 0.3 g K₂H₂PO₄, 0.5 g MgSO₄.7H₂O, and 1.0 g Instant Ocean. Cultures were incubated on orbital shakers at 140 rpm and 26-27° C. Culture broths were harvested by centrifugation. Galactosidase activity was determined using p-nitrophenol-α-D-galactopyranoside (PNPG, Sigma) as substrate in acetate buffer (0.05M), pH 4.0 (unless otherwise stated) and incubated for 1 hour at 37° C. The reaction was stopped by adding sodium borate buffer (pH 9.7) and the resulting OD₄₀₅ read using a Molecular Devices THERMOₘₐₓ microtiter plate reader. For pH profiles, KCl/HCl buffers were used for pH 1.2 and 1.6, citrate/phosphate buffers were used for pH 3-7 and phosphate/NaOH for the remaining pH readings. Heat stability was determined by heating the culture broth to 65° C. for 20 minutes before dilution with assay buffers.

Fungal strains ILF2175 and ILF5916 (deposited in the ATCC and identified with Accession No. ______ and No. ______) were isolated from soils using the stamping method (Hunter & Belt 1999). ILF2175 was isolated from a California desert sample, ILF5916 was isolated from a sample of milo grain dust obtained in Kansas, USA. Dry soil or grain dust was directly applied to selective agar media using a sterile foam plug. The plug was first pressed into the soil, any residual particles were shaken off and the plug was then applied to the agar thirteen times per plate (10 on the perimeter and 3 in the center). The same plug was used on the remaining agar plates, thus resulting in a dilution effect. Each soil or grain dust samples was applied to multiple media including Soil Extract, Rose Bengal, Corn Meal, and ISP2 (Hunter and Belt, 1999). Antibiotics (300 mg/L Penicillin and 100 mg/L Streptomycin) were added to inhibit bacterial growth. Stamping began with the leanest media (Soil Extract) and progressed to the richest (ISP2). Plates were incubated at room temperature and periodically observed for growth. Fungal hyphae were removed using a sterile toothpick and transferred to an appropriate agar maintenance medium (ISP2). Subsequent transfers of hyphae were performed until axenic cultures were obtained. Pure cultures were stored frozen at minus 80 C and on slants at room temperature.

### EXAMPLE 2

### Biochemical Screening of Soil Samples

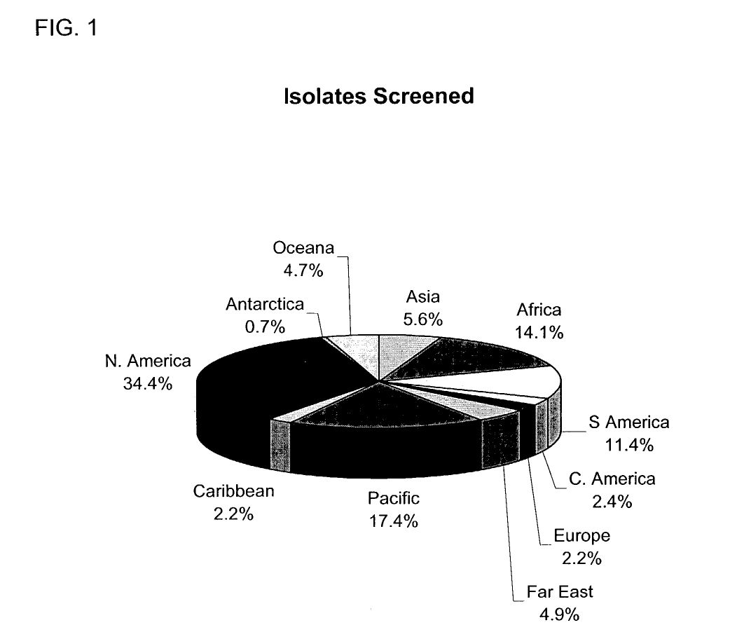

Fungi were isolated from a broad range of geographic origins and environmental conditions to provide a broad sampling of genetic diversity (FIG. 1). The primary screen involved growing the isolates in liquid broth medium and testing the supernatant for galactosidase activity remaining after heating at 65° C. for 20 minutes. More than 30 isolates passed the primary screen and were characterized in more detail. These heat stable isolates also represented a broad diversity of geographical origins and habitats (Table 1).

TABLE 1
Origins of Heat-stable Isolates

| COUNTRIES/STATES | HABITATS |
|---|---|
| Antarctica | Alpine Meadow |
| Australia | Delta |
| Chile | Desert |
| Costa Rica | Forest |
| Ethiopia | temperate |
| Hawaii | semi-tropical |
| Indonesia | tropical |
| Nepal | rain forest |
| New Zealand | Glacial |
| Nigeria | Hot Springs |
| Pacific Islands | Marine/Coastal |
| South Africa | Marsh |
| USA: CA, CO, IL, KS | Plant-Associated |
| MS, MO, NJ | |

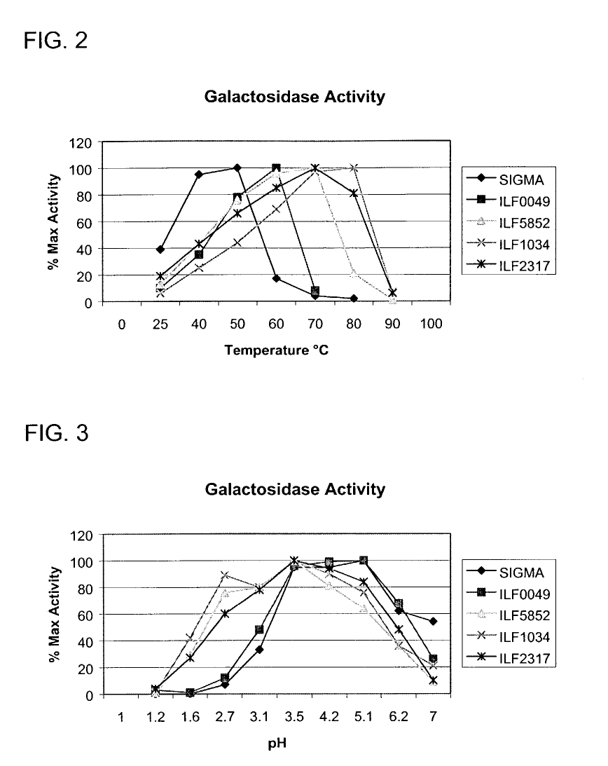

The enzyme activities that survived the 65° C. heat treatment were then tested for relative activity at different temperatures and pH. FIGS. 2 and 3 show examples of the types of profiles obtained. Some of the isolates (e.g., ILF0049) produced a temperature profile that was similar to that of a standard, commercially available enzyme obtained from SIGMA (FIG. 2), while others (e.g., ILF2317) exhibited a broader temperature profile. Some of the enzymes showed their maximum activity at temperatures as high as 80° C. (e.g., ILF1034).

The pH optimum of the SIGMA enzyme was about pH 5.0, with only less than 10% of the maximum activity below pH 3.0. Some of the experimental isolates showed a similar pH profile (e.g., ILF0049), while others (ILF5852, ILF1034 and ILF2317) retained high activity below pH 3.0 and significant activity at pH 1.6.

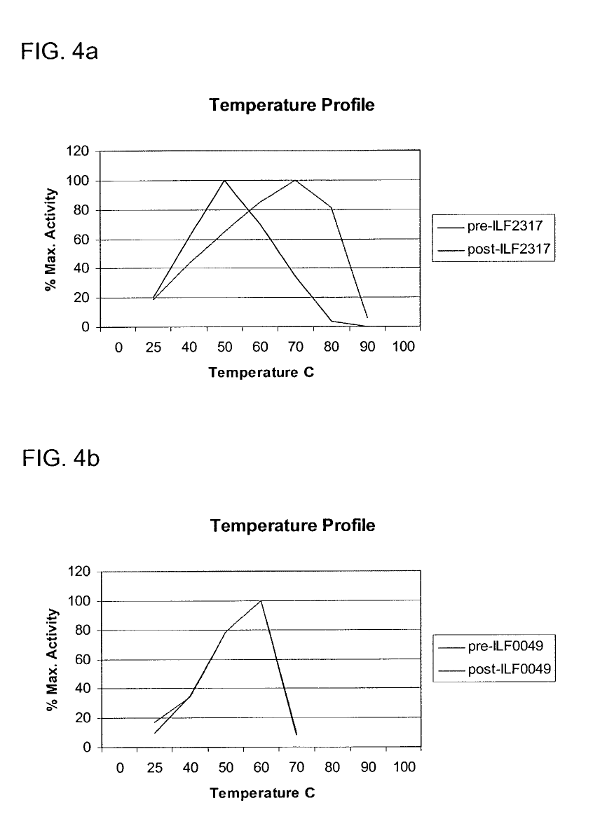

Although a number of isolates produced enzymes that were active at elevated temperatures and at low pH, the 65° C. heat treatment caused losses of more than 50% and up to 100% of the activity in the non-heated samples. These losses could be due to partial loss of activity of the enzyme(s) characterized after heat treatment, or total loss of heat-sensitive enzymes leaving only heat-stable enzyme(s) remaining. Data suggestive of both types of situation were observed. Analysis of temperature profiles of the enzyme activities in culture broths pre- and post-heat treatment (FIG. 4a) showed some examples where the profile was unchanged (e.g., ILF 0049), consistent with partial loss of activity of the major enzyme component(s). In contrast, the temperature profiles for ILF 2317 (FIG. 4b) suggest that the enzyme that survived the 65° C. heat treatment was different from the major (99%) galactosidase(s) produced by this isolate.

Data on strains identified in Experiment 1 as producing galactosidase activity which survived heat treatment at 65° C., had temperature optima as high as 89° C., retained significant activity at physiological temperatures, and had high activity below pH 3.0 are summarized in Table 2. These results suggest that it may be possible to produce large amounts of a galactosidase enzyme that combines a plurality of improved characteristics by appropriate screening of soil samples collected over diverse geographic areas with diverse environments.

TABLE 2
Summarized Properties of the Best Performers

| | % 65° C. | T | % max at | % max at |
|---|---|---|---|---|
| Sample | stable | max | 40° C. | pH 2.7 |
| Sigma | 0 | 50 | 95 | 7 |
| ILF0049 | 36 | 60 | 35 | 12 |
| ILF2317 | 1 | 70 | 43 | 76 |
| ILF5852 | 10 | 70 | 43 | 76 |
| ILF1034 | 11 | 80 | 25 | 89 |

### EXAMPLE 3

### Analysis of Activity Under Standard Conditions

Biochemical analysis of fibrolytic enzymes, e.g., α-galactosidase and endocellulase, in a total of 13 fungal strains that were pre-selected using the microbial screening techniques previously described using selected soil samples from a large soil sample collection. α-Galactosidase facilitates the release of additional nutrients by hydrolyzing galactosides contained in soybean meal into galactose and sucrose. Endocellulase and xylanase seem to primarily reduce the viscosity of the soluble fiber in animal digesta as well as releasing free sugars that can readily be absorbed and metabolized by the host animal. Enzymes secreted into the culture medium from microorganisms were investigated for the following: enzymatic activities of endo-1,4-β-glucosidase, α-galactosidase, xylanase, β-glucanase; properties of thermostability, activity optimum, and stability at a broad pH range and temperature optimum of α-galactosidase and endo-1,4-β-glucosidase. Maximum thermostability of α-galactosidase was observed at greater than 60 minutes and 20 minutes for ILF5916 and ILF2175, respectively. Also, maximum thermostability of endo-1,4-β-glucosidase was observed at greater than 4 hours for ILF5873. Commercial enzymes used as experimental controls typically display stability in the 0.5-2 minute range. These three strains were identified to be Aspergillus. Other strains were identified as Fusarium or Penicillium. Some strains exhibited temperature optima for α-galactosidase at temperatures as high as 80° C. and broad pH maxima between 2.5 and 7. The results of this experiment suggest that microorganisms isolated from soil samples from unusual habitats, primarily Aspergillus niger, can provide genes for α-galactosidase and endo-1,4-β-glucosidase enzymes with unusual biochemical characteristics for further engineering into production hosts.

Strains and Media Strains were stored on potato dextrose or V-8 juice agar at 25° C. Three different media (A, B and C) were used for fungi cultivation:

Medium A: 1.4 g/l (NH₄)₂SO₄, 2.0 g/l KH₂PO₄, 0.3 g/l MgSO₄×7H₂O, 0.3 g/l urea, 5.0 mg/l FeSO₄×7H₂O, 1.6 mg/l MnSO₄×H₂O, 1.4 mg/l ZnSO₄×7H₂O, 2.0 mg/l CoCI₂, 0.75 g/l peptone, 02 g/l Tween 80;

Medium B: 0.3 g/l KH₂PO₄, 0.5 g/l MgSO₄×7H₂O, 0.7 g/l K₂HPO₄, 1.0 g/l yeast extract, 25 g/l soy flour, 1.0 g/l Instant Ocean;

Medium C: 7.0 g/l KH₂PO₄, 1.0 g/l (NH₄)₂SO₄, 0.1 g/l MgSO₄×7H₂O, 2.0 g/l K₂HPO₄, 0.6 g/l yeast extract.

Medium A was modified from the original Maudels-Sternberg medium (Mandels, M. and D. Strenberg (1976) J. Ferment. Technol., 54 (4): 267-286). Cultivation was conducted in two steps. Step 1 involved preparing the inoculum or seed culture. The fungi were grown for 36-48 hours (medium A), and 12-24 hours (medium C) in salt media with the addition of 20 g/l glucose as a carbon source. Step 2 consisted of transferring the inoculum into fresh salt medium in the amount of 5-10% v/v using different carbon sources. No soluble carbohydrates, except lactose in some cases, were added in the production phase. In addition to the above mentioned media, the following ingredients were used as carbon and nitrogen sources: hay, whey, wheat straw, barley, spent grain, soybean hulls, soy flour, or sugar beat pulp, corn steep powder, corn steep liquor, soybean steep liquor and Traders protein.

Thermostability for carbohydrases was examined using supernatant samples heated to 65° C. and aliquots were removed in time intervals as indicated in the graphs. The sample was filtered through Cameo 25NS, 1.2 micron and placed in a glass tube with stopper. The water bath was set 70° C. to provide 65° C. inside the tube. The sample was monitored at 65° C. with a thermometer fully submerged. It took approximately one minute to reach 65° C. Aliquots were removed and placed immediately on ice to stop inactivation of the enzyme. Thermostability was measured by calculating half time. Half time (τ ½) of inactivation of the enzymes assayed was determined by plotting time versus activity. Temperature optimums for α-galactosidase and endo-1,4-β-glucosidase were determined by conducting standard enzyme assays at various temperatures (22-90° C.). The pH optima were determined by conducting standard enzyme assays at various pH values (pH 3.0 to 8.0). The following buffer solutions were prepared: 0.05 M tartaric buffer for pH 3.0 and 3.5; 0.05 M acetate buffer for pH 4.0, 5.0 and 5.5; 0.05 M potassium phosphate buffer for pH 6.0, 6.5, and 7.0; and 0.05 M tris-HCL buffer for pH 8.0. The pH-stability was determined by measuring the activity levels of the sample at pH 3.0.

Enzymatic Activities For the α-galactosidase activity assay, enzyme and substrate solutions were preheated separately for 5 minutes at 37° C. The reaction mixture contained 2 ml of 1.2-mM p-nitrophenyl-α-D-galactopyranoside (PNPG) (Sigma) in 0.05 M sodium acetate, pH 5.5, and 1 ml of enzyme sample were incubated at 37° C. for 15 minutes. All reactions were initiated by the addition of substrate. The reagent blank contained 2 ml of PNPG and 1 ml buffer. The linear range was between 0.1 to 1.5 optical density units. The reaction was stopped by the addition of 5 ml of 0.0625 Borax-NaOH, pH 9.7. The liberated p-nitrophenol was measured spectrophotometrically at 405 nm using a Beckman DU 640 instrument. One unit of α-galactosidase activity is defined as the amount of the enzyme that releases 1 μmol-p-nitrophenol from p-nitrophenyl-α-D-galactopyranoside per minute under the given reaction conditions. The molar extinction coefficient of p-nitrophenol is 18300 M⁻¹ cm⁻¹.

For the endo-1,4-β-glucosidase activity assay, enzyme and substrate solutions were preheated separately for 5 minutes at 50° C. The reaction mixture contained 3 ml of 2% carboxymethyl cellulose (CMC, Sigma) in 0.05M sodium acetate buffer, pH 4.5, and 20-100 μL of appropriately diluted enzyme solution. The mixture was incubated at 50° C. for 20 minutes. After incubation, 1 ml of the reaction solution was removed and 1 ml of p-hydroxybenzoic acid hydrazide (Sigma) (Lever, M. (1972) Analytical Biochemistry, 47: 273-279) was added. The solution was boiled for 10 minutes and then cooled for 5 minutes. Optical density was read at 410 nm. A standard curve was prepared using 0.011 to 0.055 μM glucose in 0.05M sodium acetate buffer, pH 4.5. The reagent blank contained 3 ml of 2% CMC and 20-100 μL buffer solution. The sample blank contained 3 ml buffer and 20-100 μL of enzyme. One unit of endo-1,4-β-glucosidase activity is defined as the amount of enzyme that releases 1 μmol of reducing sugar measured as glucose per minute under the given reaction conditions.

Protein concentrations were measured by the Lowery method using a commercial protein assay reagents (Sigma) according to the supplier's instructions based on bovine serum albumin as a standard.

### Results

The majority of the microorganisms advanced from Experiment 1 were grown in two media specifically developed for the genera Trichoderma (Medium A) and Aspergillus (Medium C).

Table 3 compares endoglucanase and α-galactosidase activities secreted by all fungal strains investigated under standard conditions. Most strains were tested on two different media (Media A and C).

TABLE 3
Comparative Study Of α-Galactosidase
and endo-1,4-β-glucosidase Activities

| | | Protein | α-gal | Endo |
|---|---|---|---|---|
| Strains | Media | g/l | IU/ml | IU/ml |
| ILF 2102 | Medium A | 1.3 | 0.1 | 7.8 |
| | Medium C | 6.7 | 0.4 | 13.0 |
| ILF 2175 | Medium A | 0.8 | 0.8 | 0.01 |
| | Medium C | 4.8 | 1.6 | 9.4 |
| ILF 2935 | Medium A | 0.8 | 0.8 | 0.01 |
| | Medium C | 3.0 | <0.01 | <0.01 |
| ILF 5380 | Medium A | 1.0 | 0.10 | 0.10 |
| | Medium C | 3.2 | 0.20 | 0.70 |
| ILF 5655 | Medium A | 1.30 | 0.10 | 0.20 |
| | Medium C | 2.40 | 0.01 | 0.11 |
| ILF 5672 | Medium A | 2.0 | 0.70 | 0.2 |
| | Medium C | 4.80 | | 3.8 |
| ILF 5676 | Medium A | 0.70 | 0.30 | 0.40 |
| | Medium C | 0.70 | 0.90 | <0.01 |
| ILF 5691 | Medium A | 2.2 | 2.3 | 7.7 |
| | Medium C | 6.3 | 3.5 | 2.3 |
| ILF 5701 | Medium A | 1.2 | <0.1 | 1.7 |
| | Medium C | 3.9 | <0.1 | ND¹ |
| ILF 5712 | Medium A | 1.60 | 1.70 | 4.20 |
| | Medium C | 1.10 | 0.05 | <0.01 |
| ILF 5835 | Medium A | 1.1 | 0.12 | 0.4 |
| | Medium C | 1.9 | <0.10 | ND¹ |
| ILF 5873 | Medium C | 1.00 | 4.00 | 4.6 |
| ILF 5916 | Medium C | 3.4 | 0.8 | 12.0 |

¹No activity detected

A wide range of activities was observed for both, x-galactosidase and endo-1,4-β-glucosidase. A high level of α-galactosidase activity was observed in strain ILF 5873 (4.0 IU/mL) and ILF 5691 (3.5 IU/mL). Intermediate activities were produced by ILF 2175 (1.6 IU/mL), and ILF 5916 (0.8 IU/mL). For the majority of α-galactosidase producers, low activities were observed. Generally, medium C provided a more favorable environment for increased levels of α-galactosidase activity in Aspergillus spp., especially ILF 5873, when compared to medium A. ILF 5835 and ILF 5691 likely would produce better activity if cultivated in a medium specialized for Penicillium spp.

As for the endo-1,4-β-glucosidase activities, with a high activity of 12.0 IU/ml and a low activity of 0.01 IU/ml a wide range of activities was observed again. High levels of endo-1,4-β-glucosidase activity for ILF 5691 (7.7 IU/ml versus 5.8 IU/ml) were observed. Elevated levels of activity were also observed in ILF 2102 (13 IU/mL), ILF 5916 (12 IU/mL) and ILF 2175 (9.4 IU/mL). Strains with higher activities in this study belong mostly to the genus Aspergillus and Penicillium. ILF 2175 expressed a balanced combination of both α-galactosidase and endo-1,4-β-glucosidase activity.

In summary, ILF 2175 and ILF 5916 are the most promising microorganisms for further investigation. Strains ILF 5873 and ILF 5691 also produce a balance of several enzymatic activities.

### EXAMPLE 4

### Evaluation of Thermostability, Temperature Optima, and pH Optima

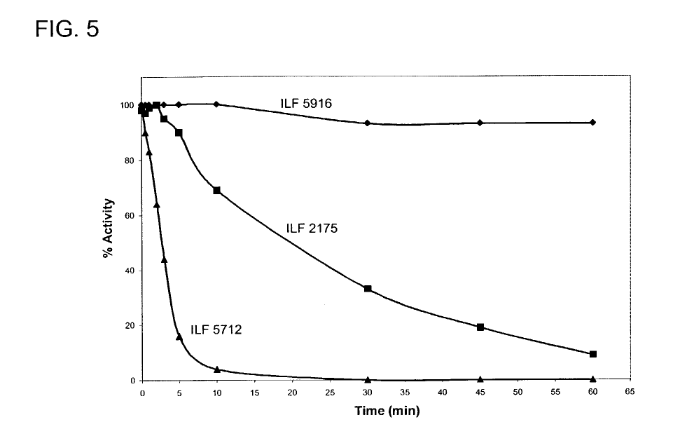

For the determination of the thermostability of α-galactosidase and endo-1,4-β-glucosidase in the fungal strains we chose as measurement the half time of inactivation at 65° C., e.g., the time when an enzyme lost 50% of its initial activity. The temperature of 65° C. for the investigation of thermostability was chosen because this is the pasteurization temperature and it is a commonly used temperature in the scientific literature. The results of the study are presented in Table 4. Table 5 compares results obtained under the conditions described above and results obtained using an assay which measured the percentage of remaining activity after a 20 minute incubation at 65° C. FIG. 5 displays the thermostability plots for three selected strains.

TABLE 4
Comparative Study of Thermostability at 65° C.

| | | endo-1,4-β- |
|---|---|---|
| | α-Galactosidase | glucosidase |
| ILF | Activity | Activity |
| Strains | t½ (min) | T½ (min) |
| 5873 | <0.5 | 271 |
| 5835 | <0.5 | — |
| 5672 | <0.5 | 1.42 |
| 5712 | 2.7 | 4.8 |
| 5916 | 117 | — |
| 2102 | <0.5 | — |
| 2175 | 20 | — |
| 2935 | ND¹ | — |
| 5380 | — | ND¹ |
| 5655 | — | 7.0 |
| 5676 | — | 2.3 |
| 5691 | — | 15 |
| 5701 | — | <0.5 |

¹ND - No activity detected

As can be seen from Table 4, the half-life for α-galactosidases activity was generally low at around 0.5 min. Selected samples are given in FIG. 5. In particular, two strains, ILF 2175 and ILF 51916, revealed high thermostability properties. Their half-life for α-galactosidase activity was 20 minutes and 117 minutes, respectively. For comparison, the α-galactosidase activity in two commercial products from Novo, 600 L and 1000 L, have a τ ½ of less than 1 minute and 18 minutes, respectively.

The themostability of endo-1,4-β-glucosidase was more variable as compared to α-galactosidase. In particular, one strain, ILF 5873, displayed dramatically enhanced thermostability with τ ½ of 271 minutes. Study of thermostability of xylanases from strains ILF 2102, 5712, 2175 and 5916 did not reveal thermotolerant enzymes (τ ½ less than 60 second; data not shown).

Table 5 displays the temperature optima for all strains that were advanced from Experiment 1. Generally, optima are shifted towards higher temperatures as compared to standard enzymes from commercial sources. In good agreement with the thermostability results, strains ILF 5916 and also ILF 5672 display the highest temperature optima of 80° C.

TABLE 5
Temperature Optima

| | | α-Galactosidase | Endoglucanase |
|---|---|---|---|
| ILF Stains | Taxonomy | (° C.) | (° C.) |
| 5873 | Aspergillus | 60 | 60 |
| 5835 | Penicillium | 60 | — |
| 5672 | Aspergillus | 80 | 60 |
| 5712 | Penicillium | 60 | 60 |
| 5916 | Aspergillus | 80 | — |
| 2102 | Actinomycetes | 60 | — |
| 2175 | Unclassified | 60 | — |
| 2935 | Aspergillus | 60 | — |
| 5380 | Unclassified | — | 50 |
| 5676 | Unclassified | — | 50 |
| 5691 | Penicillium | — | 60 |
| 5701 | Unclassified | — | 30 |

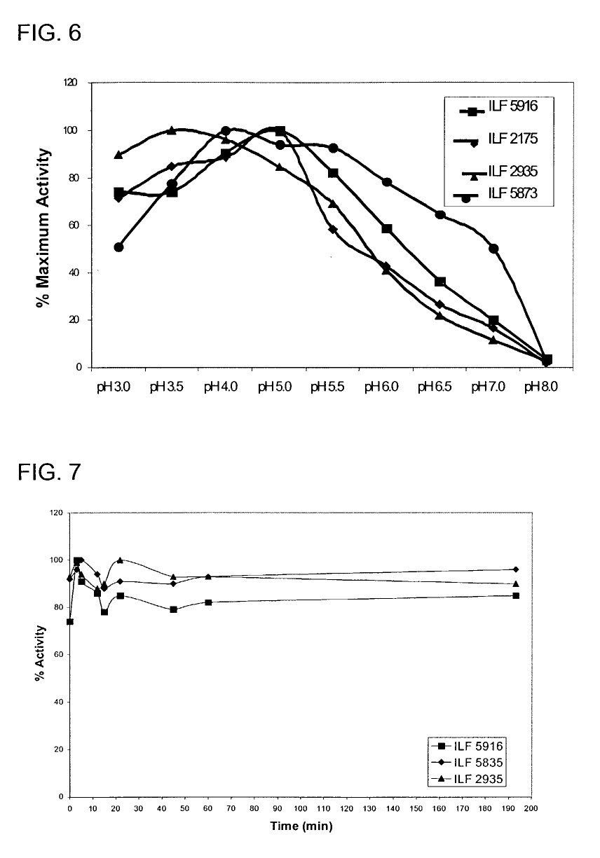

The results of the determination of the pH optima and pH range of activity for α-galactosidase for the strains are presented in Table 6. Many strains displayed broad pH maxima rather than a sharp activity peak. Selected examples are given in FIG. 6. The desired pH optimum range for animal applications is between pH 2 and 6.0, while other industrial uses of enzymes require activity at alkaline pH values. Several strains displayed significant activity at pH 3. Additionally, the stability of α-galactosidase at a lower pH of 3.0 was measured for several strains. As can be seen from FIG. 7, ILF 5916, ILF 5835 and ILF 2935 were stable at pH 3.0 for more than 2 hours

TABLE 6
pH Optima for α-Galactosidases

| ILF Stains | Taxonomy | α-gal |
|---|---|---|
| 5873 | Aspergillus | 4.0 |
| 5835 | Penicillium | 4.0-5.0 |
| 5672 | Aspergillus | 5.0-5.5 |
| 5712 | Penicillium | 4.0-5.0 |
| 5916 | Aspergillus | 5.0 |
| 2102 | Actinomycetes | 5.0 |
| 2175 | Aspergillus | 5.0 |
| 2935 | Aspergillus | 3.5-4.0 |
| 5691 | Penicillium | 5.0 |
| 5380 | Unclassified | 5.0-5.5 |

### EXAMPLE 5

### Evaluation of Xylanase Activity

For the xylanase activity assay, enzyme and substrate solutions were preheated separately for 5 minutes at 50° C. The reaction mixture contained 1 mL of 0.5% birchwood xylan (Sigma) in 0.05 M citric acid buffer, pH 5.3, and 1 ml of diluted enzyme. The mixture was incubated for 15 minutes at 50° C. The assay was continued by the addition of Somogyi-Nelson reagents (Lindner, William (1988) Methods in Enzymology, 160: 376-37). The samples were placed in a boiling water bath for 20 minutes and then cooled to room temperature. The addition of 1 ml of arsenomolybdate reagent led to a color reaction and optical density was read at 540 nm. A standard curve was prepared using 0.266 to 1.065 μM xylose in 0.05 M citric acid buffer, pH 5.3. The reagent blank contained 1 ml of xylan and 1 ml of buffer solution. The sample blank contained 1 ml buffer and 1 ml of enzyme. One unit of xylanase activity is defined as the amount of enzyme that releases 1 μmol-of reducing sugar measured as xylose per minute under the given reaction conditions. Concentrated culture supernatant samples were incubated at 65° C. with aliquots removed at the times listed in Table 8 and assayed for residual activity.

The data presented shows four distinct trends, a complete elimination of activity, a complete retention of activity, a linear decrease in activity and an initial decline followed by a leveling off of activity. These trends indicate there are enzymes that have no thermotolerance, complete thermotolerance, limited stability over time and perhaps two enzymes in a single sample with xylanase activity with one being non-thermotolerant and another retaining activity over time. The temperature of 65° C. for the investigation of thermostability was chosen because this is the pasteurization temperature and it is a commonly used temperature in the scientific literature. The results of the study are presented in Table 8. Table 8 sets out the thermostability plots for ten selected strains of fungi, actinomycetes and bacteria.

TABLE 8
Comparative Study of Xylanase Thermostability at 65° C.

| | Xylanase Activity | | | | | | | |
|---|---|---|---|---|---|---|---|---|
| | Units of activity remaining after indicated time: | | | | | | | |
| | | | | 15 | 20 | 25 | 30 | 60 |
| IL Strains | 0 min | 5 min | 10 min | min | min | min | min | min |
| ILA10905A | 0.88 | 0.81 | 0.78 | 0.80 | 0.83 | 0.72 | 0.83 | 0.82 |
| ILA11316A | 1.7 | 2.3 | 1.5 | 1.4 | 1.4 | 1.3 | 1.3 | 1.4 |
| ILB13326B | 26 | 26 | 32 | 27 | 28 | 32 | 30 | 32 |
| ILB17362B | 13 | 13 | 12 | 11 | 14 | 13 | 13 | 14 |
| ILF13735B | 5.5 | 1.2 | 1.2 | 1.0 | 1.1 | 0.6 | 0.7 | 0.4 |
| ILA10011A | 1.6 | 1.2 | 0.9 | 0.9 | 0.8 | 0.8 | 0.9 | 0.8 |
| ILB15158B | 13 | 13 | 14 | 14 | 16 | 16 | 16 | 16 |
| ILA10957A | 50 | 50 | 14 | 20 | 9 | 6 | 4 | 2 |
| ILF13735A | 15 | 13 | 7 | 7 | 6 | 6 | 6 | 3 |
| ILF16631 | 84 | 84 | 16 | 17 | 16 | 16 | 14 | 10 |

### Discussion

The objective of the experiments was to identify microbial strains producing α-galactosidase, endocellulase, and xylanase enzymes with enhanced properties such as thermostability, prolonged shelf life, resistance against proteases and activity at low pH values for use as feed and food additives. In this study we only analyzed for thermostability, temperature optima and pH maxima. For industrial scale production, recombinant DNA technologies in combination with fermentation technology could be used, as could other methods known in the art. The data indicate that two strains expressing α-galactosidase and at least six strains expressing endo-1,4-β-glucosidase are very promising candidates for further investigation, e.g., purification of the protein and cloning of the gene. According to our investigations ILF 2175 and ILF 5916, which are Aspergillus niger and Aspergillus fumigatus, are able to produce thermostable α-galactosidases, in addition to cellulases, xylanases, and β-glucanases. Strain ILF 5873 produces a highly thermostable endo-1,4-β-glucosidase. Thermostabilities are significantly enhanced over more commonly found stabilities. In particular, the role of proteases and naturally occurring stabilizers in the crude homogenates used for the analysis need to be examined. However, the results effectively validate the approach to screen microbial diversity in soil samples for enzymes with unusual properties. In the past, microbes from extreme environments have been screened for similar purposes. In those cases enzymes were identified that almost exclusively display activity profiles with little or no activity at room temperature or slightly above.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

We claim:

1. A method for identifying an enzyme of a desired activity produced by microorganisms that have improved activity under two or more extreme conditions, comprising the steps of:
a. assembling a library of soils and other environmental samplesfrom a variety of geographic locations having diverse environmental conditions;
b. isolating and purifying genetically diverse microorganisms using a range of selective isolation conditions;
c. screening the library for microorganisms that produce enzymes having the desired activities and properties;
d. screening the isolated microorganism for activity under two or more selected extreme conditions to identify a microorganism that produces an enzyme having improved activity under the two or more extreme conditions.

2. The method as defined in claim 1, wherein the two or more extreme conditions are selected from the group comprising temperature activity maxima higher than commonly found, temperature activity minima lower than commonly found, pH activity minima lower than commonly found, pH activity maxima higher than commonly found, improved thermostability, extended shelf-life, and high activity at typical animal body temperatures

3. The method as defined in claim 1, wherein the microorganisms are selected from the group comprising bacteria and fungi.

4. The method as defined in claim 3, wherein the fungi are selected from the group comprising Aspergillus, Fusarium, Penicillium, and Trichoderma.

5. The method as defined in claim 1, wherein the enzyme was selected from the group consisting of cellulases, galactosidases, glucanases, glucosidases, and xylanases.

6. The method as defined in claim 1, wherein an extreme condition is an enzymatic activity half-life of between about 15 minutes and about 300 or more minutes at a minimum temperature of 65° C.

7. The method as defined in claim 1, wherein an extreme condition is a temperature optimum greater than about 50° C.

8. The method as defined in claim 6, wherein a second extreme condition is a temperature optimum greater than about 50° C.

9. The method as defined in claim 1, wherein an extreme condition is a pH stability of more than about 2 hours at pH 3.0.

10. The method as defined in claim 6, wherein an extreme condition is a pH stability of more than about 2 hours at pH 3.0.

11. The method as defined in claim 8, wherein a third extreme condition is a pH stability of more than about 2 hours at pH 3.0.
